(19) United States
(12) Patent Application Publication
(10) Pub. No.: US 2020/0113895 A1
(43) Pub. Date: Apr. 16, 2020

(54) Method of treating peripheral neuropathy

(71) Applicant: Chemedest Ltd., Tartu (EE)

(72) Inventors: Mati Karelson, Tartu (EE); Mart SAARMA, Tartu (EE); Neinar SELI, Tartu (EE)

(73) Assignee: Chemedest Ltd., Tartu (EE)

(21) Appl. No.: 16/713,033

(22) Filed: Dec. 13, 2019

Related U.S. Application Data
(63) Continuation of application No. 15/682,864, filed on Aug. 22, 2017.
Application No. 15/682,864 is a continuation of application No. 14/899,060, filed on Dec. 16, 2015, now abandoned.
(60) Provisional application No. 61/702,085, filed on Sep. 17, 2012.

(51) Int. Cl. A61K 31/496; A61K 9/00; A61K 31/495; A61K 31/473; A61K 31/4706; A61K 31/433; A61K 31/429; A61K 31/423; A61K 31/415; A61K 31/381

(52) U.S. Cl. CPC A61K 31/496; A61K 9/0014; A61K 31/495; A61K 31/473; A61K 31/381; A61K 31/433; A61K 31/429; A61K 31/423; A61K 31/415; A61K 31/4706

(57) ABSTRACT

Compositions and methods for treating or preventing peripheral neuropathy in a subject determined to be in need thereof. The method comprises a step of topically administering to the subject anti-peripheral neuropathic compounds acting as GFR α 3 type receptor agonist.

### PRIORITY

This application is a continuation application of U.S. application Ser. No. 15/682,864 filed on Aug. 22, 2017 which is a continuation of U.S. application Ser. No. 14/899,060 filed on Dec. 16, 2015 claiming priority of PCT application PCT/EP2013/069184 filed on Sep. 16, 2013, claiming priority of U.S. provisional application 61/702,085 filed on Sep. 17, 2012, the contents of all of which are incorporated herein by reference.

### TECHNICAL FIELD

A method of treating or preventing peripheral neuropathy in a subject determined to be in need thereof comprising: topically administering to the subject an anti-peripheral neuropathic compound acting as GFR α 3 type receptor agonist.

### BACKGROUND ART

Many neurological and all neurodegenerative diseases including peripheral neuropathy are caused by death of neurons or loss of their neuritis. Currently, there are no drugs that are neuroprotective or neurorestorative. Several proteins supporting neuronal survival have been shown to be effective against neurological and neurodegenerative diseases in animal models and GDNF family of ligands (GLFs) in chronic pain. However, proteins are large molecules with poor pharmacokinetic properties. Currently available therapies for neuropathic pain are symptomatic. The neurotrophic factor-based therapies are very promising, because in addition to the promotion of neuronal survival they also induce axonal regeneration, support the formation of synapses and stimulate functional properties of neurons.

A treatment for neuropathic pain is an important unmet medical need because this pain often is refractory to many medical interventions. An important element in the development of neuropathic pain is a dysfunction in the activity of peripheral nerves. Because neurotrophic factors affect nerve development and maintenance, modulating the activity of these factors can alter neuronal pathophysiology and produce a disease-modifying effect. Blocking the activity of nerve growth factor or enhancing the activity of either glial-derived neurotrophic factor (GDNF) or GDNF family ligand artemin (ARTN) has shown potential for normalizing neuronal activity and attenuating signs of neuropathic pain in animal models and clinical studies (Ossipov, 2011).

Thus, ARTN was found to promote the survival of several different peripheral neuron populations, including those present in the dorsal root, trigeminal, nodose, and superior cervical ganglia, as well as cultured fetal ventral mesencephalic DA neurons (Baloh et al., 1998). ARTN is a distant member of the transforming growth factor β superfamily and a member of the GDNF family ligands (GFL). This family consists of four members: glial cell line-derived neurotrophic factor (GDNF), neurturin (NRTN), artemin (ARTN) and persephin (PSPN) (FIG. 1), all of which are potent neurotrophic factors (Airaksinen and Saarma, 2002).

It has been show that systemic artemin administration in animals produced essentially complete and persistent restoration of nociceptive and sensorimotor functions, and could represent a promising therapy that may effectively promote sensory neuronal regeneration and functional recovery after injury (Wang, et al 2003).

Because the GFR α 3 receptor (the GFR α 3 is an α-subunit receptor, a receptor that complexes with a beta subunit receptor in response to ligand binding), unlike the GFR α 1 receptor, is limited in its distribution to the peripheral nervous system, artemin may produce its neuroprotective effects without the potential for the troubling adverse effects seen with GDNF. The repeated injection of artemin to rats with SNL (spinal nerve ligation) produced a dose-dependent normalization of behavioral responses to light touch and noxious thermal stimuli without producing behavioral signs of toxicity (Garden et al, 2003).

### DISCLOSURE OF INVENTION

The present invention is related to a method of treating a disorder (peripheral neuropathy) that can be treated by contacting, activating a GFR α/3RET receptor complex in a subject in need of treatment thereof, comprising administering to the subject an effective amount of a compound having binding and/or modulation specificity for a GFR α 3 receptor molecule, thereby treating the disorder. RET—rearranged during transfection.

All aspects of the invention described in relation to administering a compound or composition or substance to a subject also should be understood to relate to use of the compound or composition or substance for treatment of the subject; or for manufacture of a medicament (useful for) treatment of the condition for which the subject is in need of treatment.

Likewise, all compounds (or salts, esters, or pro-drugs thereof) described herein as useful for these purposes are themselves an aspect of the invention. Similarly, compositions comprising one or more of these compounds and a pharmaceutically acceptable diluent, excipient, or carrier, are an aspect of the invention. Similarly, unit dose formulations of one or more of the compounds are an aspect of the invention. Additionally, a medical device such as a syringe that contains the compound or composition is an aspect of the invention.

Also disclosed are the compounds, or salts or esters thereof, which can activate the GFR α 3/RET receptor complex.

In jurisdictions that forbid the patenting of methods that are practiced on the human body, the following restrictions are intended: (1) the selecting of a human subject shall be construed to be restricted to selecting based on testing of a biological sample that has previously been removed from a human body and/or based on information obtained from a medical history, patient interview, or other activity that is not practiced on the human body; and (2) the administering of a composition to a human subject shall be restricted to prescribing a controlled substance that a human subject will self-administer by any technique (e.g., orally, inhalation; topical application, injection, insertion, etc.); or that a person other than the prescribing authority shall administer to the subject. For each jurisdiction, the broadest reasonable interpretation that is consistent with laws or regulations defining patentable subject matter is intended. In jurisdictions that do not forbid the patenting of methods that are practiced on the human body, the selecting of subjects and the administering of compositions includes both methods practiced on the human body and also the foregoing activities.

### BRIEF DESCRIPTION OF DRAWINGS

The above and other objects, features and other advantages of the present invention will be more clearly understood from the following detailed description taken in conjunction with the accompanying drawings, in which:

FIG. 1 is GDNF family ligands of GFR α receptors.

FIG. 2 is paw withdrawal thresholds pre-ligation for ipsilateral and contralateral hind paws. Data are presented as mean±SEM (Scanning electron microscope);

FIG. 3 is Ipsilateral paw withdrawal thresholds following chronic administration of test compounds. Data are presented as mean±SEM Scanning electron microscope). Asterisks (**p<0.01, *p<0.05) indicate a significant difference compared to vehicle;

FIG. 4 is paw withdrawal thresholds pre-ligation for ipsilateral and contralateral hind paws. Data are presented as mean±SEM;

FIG. 5 is ipsilateral paw withdrawal thresholds following chronic administration of test compound. Data are presented as mean±SEM. Asterisks (**p<0.01, ***p<0.001) indicate a significant difference compared to vehicle;

FIG. 6 is paw withdrawal thresholds pre-ligation for ipsilateral and contralateral hind paws. Data are presented as mean±SEM.

FIG. 7 is ipsilateral paw withdrawal thresholds following chronic administration of test compound. Data are presented as mean±SEM. Asterisks (**p<0.01, ***p<0.001) indicate a significant difference compared to vehicle.

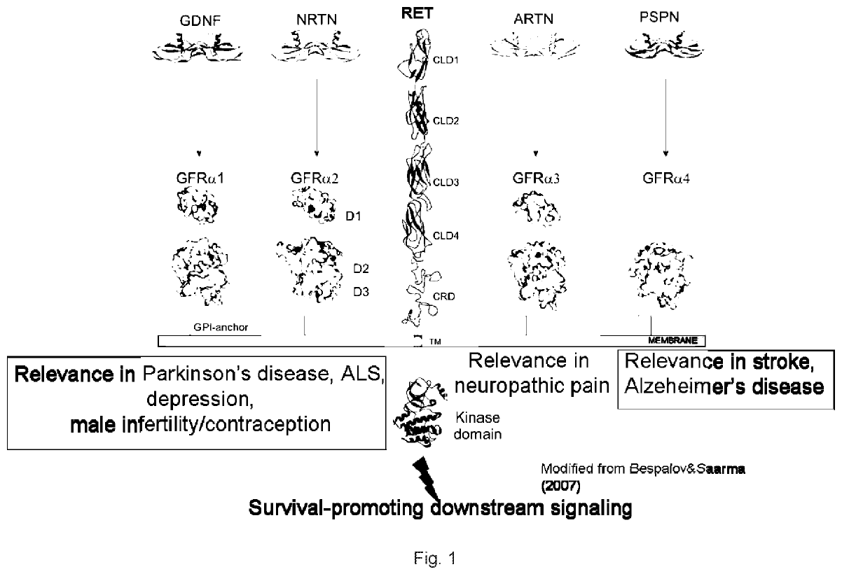

### BEST MODE FOR CARRYING OUT THE INVENTION

Disclosed herein are compounds and methods of treating a disorder (neuropathic pain) in a subject, comprising administering to the subject an effective amount of a compound having binding and/or modulation specificity for a GFR α 3 receptor molecules (“ARTN mimetic compounds”) or downstream RET signaling (“RET signaling activating compounds”). In some variations of the invention, the compound is administered in a composition that also includes one or more pharmaceutically acceptable diluents; adjuvants, or carriers.

For purposes of the disclosure, treating is considered a success if any of the following therapeutic goals are achieved: symptoms of the disease are ameliorated, alleviated, or diminished; progression of the disease or disease symptoms is slowed or arrested; deterioration or injury is alleviated, partially healed, or fully healed; and/or if the subject makes a partial or complete recovery; and/or other standard-of-care therapies that are more expensive, more difficult to administer, or have less acceptable side-effects can be reduced or eliminated while achieving a similar quality of life.

The disorder is peripheral neuropathy. The subject can be an animal or a human subject. The animal can be a mammal.

The compound can be a small molecule. In some embodiments, ARTN mimetic compound has a structure of Formula (I),

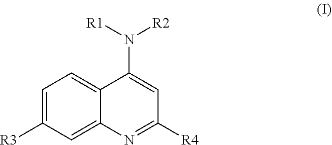

wherein R1 and R2 are independently selected from the group consisting of H, alkyl, aryl, alkylenearyl, acyl, alkoxycarbonyl, aryloxycarbonyl, alkylenearyloxycarbonyl, carbamoyl, alkylcarbamoyl, dialkylcarbamoyl, and alkyleneamino; R3 is independently selected from H, fluorine, chlorine, bromine, iodide, alkyl, aryl, alkylenearyl, acyl, alkoxycarbonyl, aryloxycarbonyl; alkylenearyloxycarbonyl, carbamoyl; alkylcarbamoyl, and dialkyl-carbamoyl and R4 is selected from the group consisting of H, alkyl, aryl, alkylenearyl, alkenylenearyl, hydroxyl; or a pharmaceutically acceptable salt thereof. In some embodiments, R1 and R2 are independently selected from the group consisting of alkyleneamino and hydrogen, where the amino group of the alkyleneamino moiety can be further substituted with one or two alkyl or alkylenearyl (e.g., a benzyl) groups. In various embodiments, R3 is chloro or aminoalkyl. In a specific embodiment, R1 is hydrogen and R2 is alkyleneamino.

In some embodiments, the GDNF mimetic compound has a structure of Formula (II),

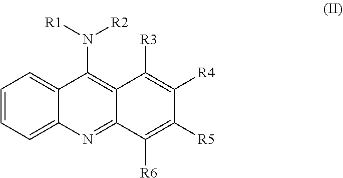

wherein R1 and R2 are independently selected from the group consisting of H, alkyl, aryl; alkylenearyl, acyl; alkoxycarbonyl, aryloxycarbonyl, alkylenearyloxycarbonyl, carbamoyl, alkylcarbamoyl, dialkylcarbamoyl, and alkyleneamino; R3, R4, R5, and R6 are independently selected from H, fluorine, chlorine, bromine, iodide, alkyl, aryl, alkylenearyl, acyl, alkoxycarbonyl, aryloxycarbonyl, alkylenearyloxycarbonyl, carbamoyl, alkylcarbamoyl, and dialkylcarbamoyl; or a pharmaceutically acceptable salt thereof.

In some embodiments, the GDNF mimetic compound has a structure of Formula (III),

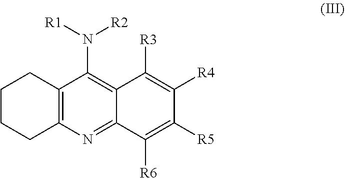

wherein R1 and R2 are independently selected from the group consisting of H, alkyl, aryl, alkylenearyl, acyl, alkoxycarbonyl, aryloxycarbonyl, alkylenearyloxycarbonyl, carbamoyl, alkylcarbamoyl, dialkylcarbamoyl, and alkyleneamino; R3, R4, R5, and R6 are independently selected from H, fluorine, chlorine, bromine, iodide, alkyl, aryl, alkylenearyl, acyl, alkoxycarbonyl, aryloxycarbonyl, alkylenearyloxycarbonyl, carbamoyl, alkylcarbamoyl, and dialkylcarbamoyl; or a pharmaceutically acceptable salt thereof.

In some embodiments, the GDNF mimetic compound has a structure of Formula (IV),

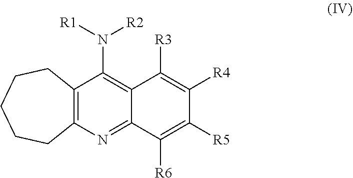

wherein R1 and R2 are independently selected from the group consisting of H, alkyl, aryl, alkylenearyl, acyl, alkoxycarbonyl, aryloxycarbonyl, alkylenearyloxycarbonyl, carbamoyl, alkylcarbamoyl, dialkylcarbamoyl, alkyleneamino; R3, R4, R5, and R6 are independently selected from H, fluorine, chlorine, bromine, iodide, alkyl, aryl, alkylenearyl, acyl, alkoxycarbonyl, aryloxycarbonyl, alkylenearyloxycarbonyl, carbamoyl, alkylcarbamoyl, and dialkylcarbamoyl; or a pharmaceutically acceptable salt thereof.

In some embodiments, the RET signaling activating compound has a structure of Formula (V),

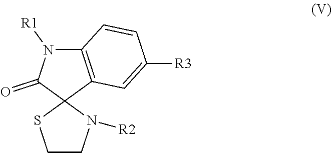

wherein R1 and R2 are independently selected from the group consisting of H, alkyl, aryl, alkylenearyl, acyl, alkoxycarbonyl, aryloxycarbonyl, alkylenearyloxycarbonyl, carbamoyl, alkylcarbamoyl, dialkylcarbamoyl, alkyleneamino; R3 is independently selected from H, fluorine, chlorine, bromine; iodide, alkyl, aryl, alkylenearyl, acyl, alkoxy, alkoxycarbonyl, aryloxycarbonyl, alkylenearyloxycarbonyl, carbamoyl, alkylcarbamoyl, and dialkylcarbamoyl; or a pharmaceutically acceptable salt thereof.

In some embodiments, the RET signaling activating compound has a structure of Formula (VI),

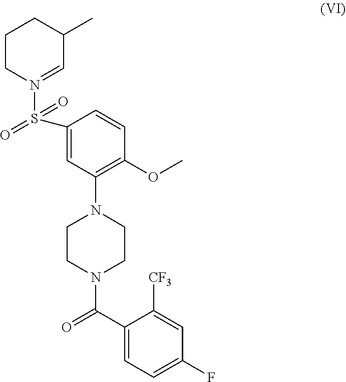

or a pharmaceutically acceptable salt thereof.

In some embodiments, RET signaling activating compound has a structure of Formula (VII),

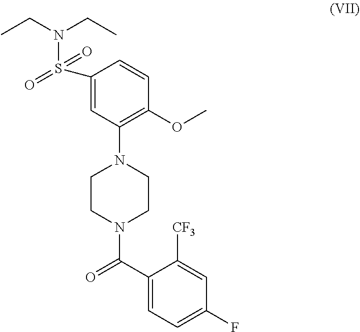 [SMILES: CCN(CC)S(=O)(=O)c1ccc(OC)c(N2CCN(C(=O)c3ccc(F)cc3C(F)(F)F)CC2)c1]

or a pharmaceutically acceptable salt thereof.

In some embodiments, the RET signaling activating compound has a structure of Formula (VIII),

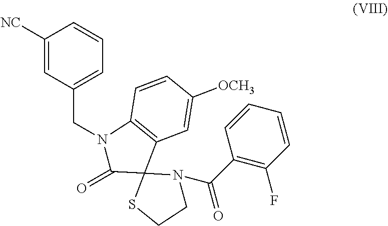 [SMILES: COc1ccc2c(c1)C1(SCCN1C(=O)c1ccccc1F)C(=O)N2Cc1cccc(C#N)c1]

or a pharmaceutically acceptable salt thereof.

In some embodiments, the RET signaling activating compound has a structure of Formula (IX),

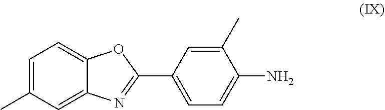 [SMILES: Cc1ccc2oc(-c3ccc(N)c(C)c3)nc2c1]

or a pharmaceutically acceptable salt thereof.

In some embodiments, the RET signaling activating compound has a structure of Formula (X),

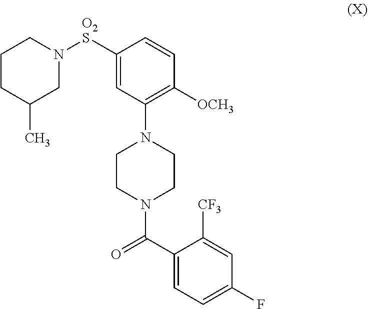 [SMILES: COc1ccc(S(=O)(=O)N2CCCC(C)C2)cc1N1CCN(C(=O)c2ccc(F)cc2C(F)(F)F)CC1]

or a pharmaceutically acceptable salt thereof.

In some embodiments, the RET signaling activating compound has a structure of Formula (XI),

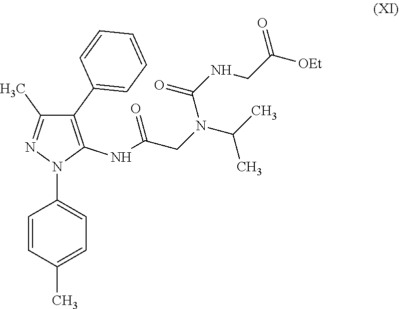 [SMILES: CCOC(=O)CNC(=O)N(CC(=O)Nc1c(-c2ccccc2)c(C)nn1-c1ccc(C)cc1)C(C)C]

or a pharmaceutically acceptable salt thereof.

In some embodiments, the RET signaling activating compound has a structure of Formula (XII),

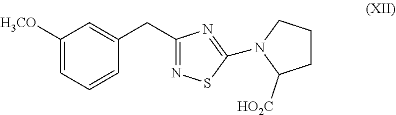 [SMILES: COc1cccc(Cc2nsc(N3CCCC3C(=O)O)n2)c1]

or a pharmaceutically acceptable salt thereof.

In some embodiments, the RET signaling activating compound has a structure of Formula (XIII),

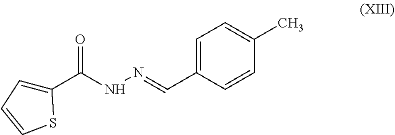 [SMILES: Cc1ccc(/C=N/NC(=O)c2cccs2)cc1]

or a pharmaceutically acceptable salt thereof.

In some embodiments, the RET signaling activating compound has a structure of any one of the following formulae:

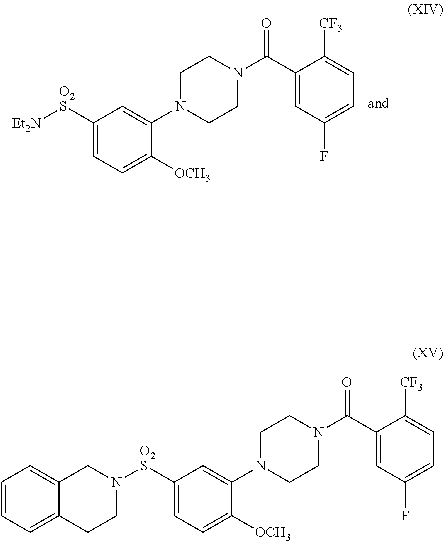

or a pharmaceutically acceptable salt thereof.

As used herein, the term “alkyl” refers to straight chained and branched hydrocarbon groups containing carbon atoms, typically methyl, ethyl, and straight chain and branched propyl and butyl groups. Unless otherwise indicated, the hydrocarbon group can contain up to 20 carbon atoms. The term “alkyl” includes “bridged alkyl,” i.e., a C6-C16 bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, amino, and sulfonyl. An “alkoxy” group is an alkyl group having an oxygen substituent, e.g., —O-alkyl.

The term “alkenyl” refers to straight chained and branched hydrocarbon groups containing carbon atoms having at least one carbon-carbon double bond. Unless otherwise indicated, the hydrocarbon group can contain up to 20 carbon atoms. Alkenyl groups can optionally be substituted, for example, with hydroxy (OH), halo, amino, and sulfonyl.

As used herein, the term “alkylene” refers to an alkyl group having a further defined substituent. For example, the term “alkylenearyl” refers to an alkyl group substituted with an aryl group, and “alkyleneamino” refers to an alkyl groups substituted with an amino group. The amino group of the alkyleneamino can be further substituted with, e.g., an alkyl group, an alkylenearyl group, an aryl group, or combinations thereof. The term “alkenylene” refers to an alkenyl group having a further defined substituent.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF3, NO2, CN, NC, OH, alkoxy, amino, CO2H, CO2alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like. An “aryloxy” group is an aryl group having an oxygen substituent, e.g., —O-aryl.

As used herein, the term “acyl” refers to a carbonyl group, e.g., C(O). The acyl group is further substituted with, for example, hydrogen, an alkyl; an alkenyl, an aryl, an alkenylaryl, an alkoxy, or an amino group. Specific examples of acyl groups include, but are not limited to, alkoxycarbonyl (e.g., C(O)—Oalkyl); aryloxycarbonyl (e.g., C(O)—Oaryl); alkylenearyloxycarbonyl (e.g., C(O)—Oalkylenearyl); carbamoyl (e.g., C(O)—NH2); alkylcarbamoyl (e.g., C(O)—NH(alkyl)) or dialkylcarbamoyl (e.g., C(O)—NH(alkyl)2).

As used herein, the term “amino” refers to a nitrogen containing substituent, which can have zero, one, or two alkyl, alkenyl, aryl, alkylenearyl, or acyl substituents. An amino group having zero substituents is —NH2.

As used herein, the term “halo” or “halogen” refers to fluoride, bromide; iodide, or chloride.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate; cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate; nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The present invention describes a method of treating or preventing peripheral neuropathy in a subject determined to be in need thereof comprising: topically administering to the subject an anti-peripheral neuropathic compound acting as GFR α 3 type receptor agonist and having one of the of the following compound structures:

compound of a structure of Formula (I)

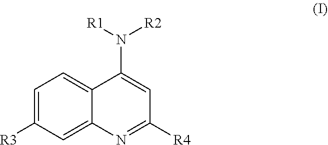

wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3 is independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl and R4 is selected from the group consisting of H, alkyl, aryl, aralkyl, hydroxyl; or a pharmaceutically acceptable salt thereof;

compound of a structure of Formula (H)

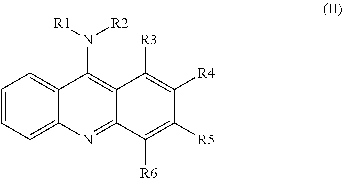

wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, R4, R5, and R6 are independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl; or a pharmaceutically acceptable salt thereof;

compound of a structure of Formula (III)

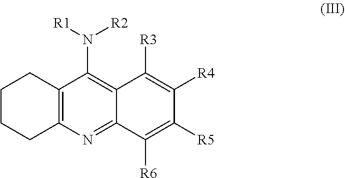

wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, R4, R5, and R6 are independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl; or a pharmaceutically acceptable salt thereof;

compound of a structure of Formula (IV)

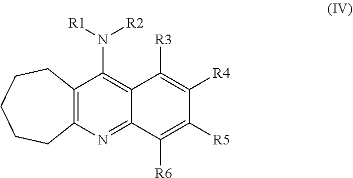

wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, R4, R5, and R6 are independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl; or a pharmaceutically acceptable salt thereof;

compound of a structure of Formula (V)

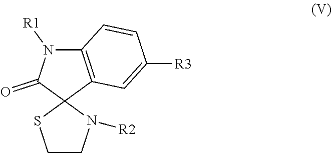

wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, is independently selected from H, fluorine, chlorine, bromine, iodide, alkyl, aryl; aralkyl, acyl, alkoxy, alkoxycarbonyl, aryloxycarbonyl, aralkoxycarbonyl, carbamoyl, alkylcarbamoyl, and dialkylcarbamoyl; or a pharmaceutically acceptable salt thereof;

The subject of the present invention is a method of treating or preventing peripheral neuropathy in a subject determined to be in need thereof comprising: topically administering to the subject an anti-peripheral neuropathic compound acting as GFR α 3 type receptor agonist and having one of the of the following compound structures:

compound of a structure of Formula (VI)

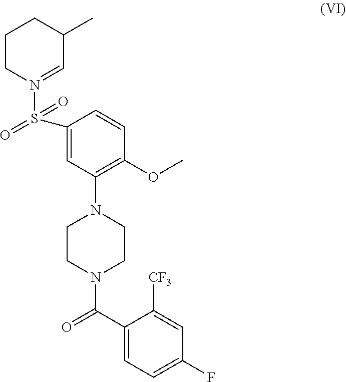

compound of a structure of Formula (VII)

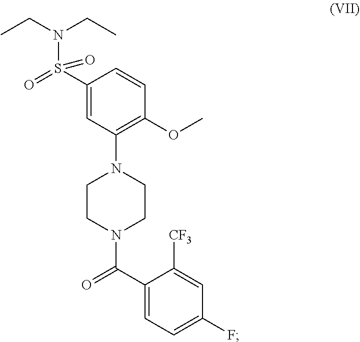 [SMILES: CCN(CC)S(=O)(=O)c1ccc(OC)c(N2CCN(C(=O)c3ccc(F)cc3C(F)(F)F)CC2)c1]

compound of a structure of Formula (VIII)

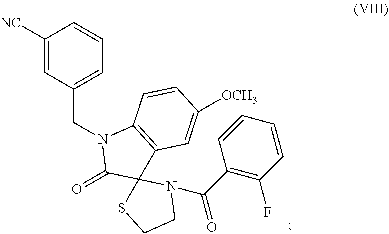 [SMILES: COc1ccc2c(c1)C1(SCCN1C(=O)c1ccccc1F)C(=O)N2Cc1cccc(C#N)c1]

compound of a structure of Formula (IX)

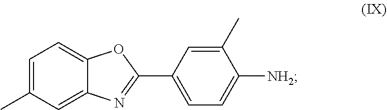 [SMILES: Cc1ccc2oc(-c3ccc(N)c(C)c3)nc2c1]

compound of a structure of Formula (X)

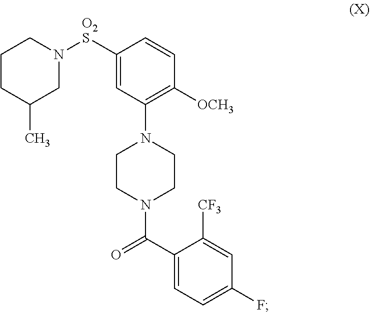 [SMILES: COc1ccc(S(=O)(=O)N2CCCC(C)C2)cc1N1CCN(C(=O)c2ccc(F)cc2C(F)(F)F)CC1]

compound of a structure of Formula (XI)

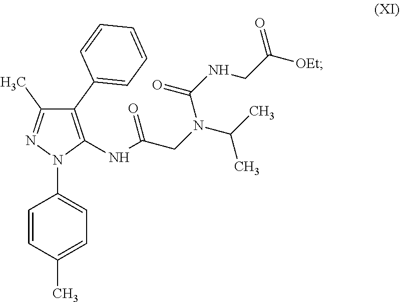 [SMILES: CCOC(=O)CNC(=O)N(CC(=O)Nc1c(-c2ccccc2)c(C)nn1-c1ccc(C)cc1)C(C)C]

compound of a structure of Formula (XII)

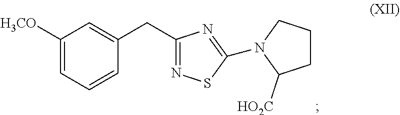 [SMILES: COc1cccc(Cc2nsc(N3CCCC3C(=O)O)n2)c1]

compound of a structure of Formula (XIII)

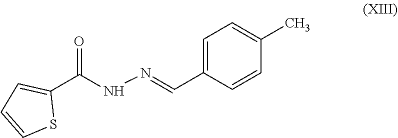 [SMILES: Cc1ccc(/C=N/NC(=O)c2cccs2)cc1]

compound of a structure of Formula (XIV)

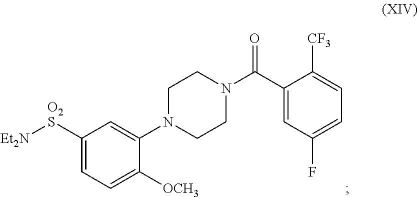

compound of a structure of Formula (XV)

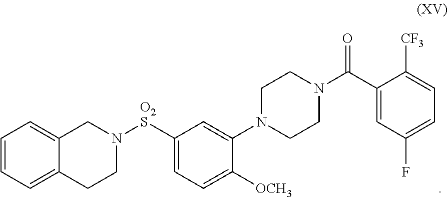 [SMILES: COc1ccc(S(=O)(=O)N2CCc3ccccc3C2)cc1N1CCN(C(=O)c2cc(F)ccc2C(F)(F)F)CC1]

### Formulations

The compounds disclosed herein can also be admixed, encapsulated, conjugated or otherwise associated with other molecules, molecule structures or mixtures of compounds, as for example, liposomes, carriers, diluents, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Further disclosed herein are pharmaceutical compositions and formulations which include the compounds described. The pharmaceutical compositions can be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal); oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The pharmaceutical formulations, which may conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions can be formulated into any of many possible dosage forms such as, but not limited to, tablets, capsules, gel capsules, liquid syrups, soft gels, suppositories, and enemas. The compositions can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension may also contain stabilizers.

Pharmaceutical compositions include, but are not limited to, solutions, emulsions, foams and liposome-containing formulations. The pharmaceutical compositions and formulations of the present invention may comprise one or more penetration enhancers, carriers, excipients, diluents, or other active or inactive ingredients.

Emulsions are typically heterogeneous systems of one liquid dispersed in another in the form of droplets usually exceeding 0.1 μm in diameter. Emulsions can contain additional components in addition to the dispersed phases, and the active drug which is present as a solution in either the aqueous phase, oily phase, or itself as a separate phase. Microemulsions are included as an embodiment of the disclosure. Emulsions and their uses are well known in the art and are further described in U.S. Pat. No. 6,287,860, which is incorporated herein in its entirety.

Formulations can include liposomal formulations. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions disclosed herein can also include surfactants. The use of surfactants in drug products, formulations and in emulsions is well known in the art. Surfactants and their uses are further described in U.S. Pat. No. 6,287,860, which is incorporated herein in its entirety.

In one embodiment, disclosed herein are formulations comprising one or more penetration enhancers to effect the efficient delivery of the compounds disclosed herein. In addition to aiding the diffusion of non-lipophilic drugs across cell membranes, penetration enhancers also enhance the permeability of lipophilic drugs. Penetration enhancers can be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the compounds of the invention are in admixture with a topical delivery agent such as lipids, liposomes, fatty acids, fatty acid esters, steroids, chelating agents and surfactants. Preferred lipids and liposomes include neutral (e.g. dioleoylphosphafidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which compounds are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds of the invention may be delivered orally, in granular form including sprayed dried particles, or complexed to form micro or nanoparticles. Complexing agents and their uses are further described in U.S. Pat. No. 6,287,860, which is incorporated herein in its entirety. Oral formulations and their preparation are described in detail in U.S. application Ser. Nos. 09/108,673, 09/315,298, and 10/071,822, each of which is incorporated herein by reference in their entirety.

Compositions and formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

### Dosing

The selection of formulations and administration (dosing) is determined, e.g., by dose-response, toxicity, and pharmacokinetic studies. Dosing is dependent on severity and responsiveness of the disease state to be treated, with the course of treatment lasting from several days to several months, or until a cure is effected, or a diminution of the disease state or disease symptoms is achieved. Dosing may continue indefinitely for chronic disease states or conditions for which diminution but no cure can be achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly; or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily; to once every 20 years.

### EXAMPLES

The following Examples have been included to provide illustrations of the presently disclosed subject matter. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### Example 1. Evaluation of the Analgesic Properties of Compounds in Rat Models of Neuropathic Pain (Bennett Model)

The study was designed to evaluate the analgesic efficacy of compound XV (CHM-65) in the Bennett model of neuropathic pain.

### 1.1 Animals

Male Sprague Dawley rats (100-125 g) from Harlan (Indianapolis, Ind.) were used in the study. Upon receipt, rats were assigned unique identification numbers and were group housed with 3 rats per cage in polycarbonate cages with micro-isolator filter tops. All rats were examined, handled, and weighed prior to initiation of the study to assure adequate health and suitability. During the course of the study; 12/12 light/dark cycles were maintained; with lights on at 7:00 am EST. The room temperature was maintained between 20 and 23° C. with a relative humidity maintained around 50%. Chow and water were provided ad libitum for the duration of the study.

### 1.2 Test Compound

Reference compound: Gabapentin (100 mg/kg; TRC, Lot No. 1-SWM-154-1) was dissolved in 0.5% carboxy-methylcellulose (CMC) in water and administered acutely on test day (day 12) 1 hour prior to testing, at a dose volume of 1 ml/kg p.o. CHM-65 (5 and 15 mg/kg, Q.D., Lot n/a) was dissolved in sterile injectable saline and administered subcutaneously at a dose volume of 3 ml/kg, on days 1, 3, 5, 8, 10, and 12, with the first administration (day 1) occurring 1 hour post-op. On day 12, compound was administered 1 hour prior to testing.

### 1.3 Methods

### 1.3.1 Chronic Constrictive Nerve Injury of the Sciatic Nerve—Bennett

This surgery was performed according to Bennett and Xie (1988). Specifically, rats were anesthetized with isoflurane (2% in air). The left hind flank was shaved and sterilized and the rat positioned on its side. The pelvic bone ridge was palpated and a vertical incision was made perpendicular to the long axis of the spine. The first layer of muscle was cut to expose the sciatic nerve. Retractors were used to open incision; centering the portion of the sciatic nerve to be ligated. The exposed nerve was carefully teased apart from the second layer of muscle, removing fascia lining, Once the nerve was freed, hooked forceps were carefully passed underneath the nerve in order to pass 5 cm lengths of 4.0 chromic gut suture under the nerve (sutures are pre-soaked in saline to ensure softness). Sutures were positioned superior to the point where the nerve branches. Each length of suture was used to make a loose ligation around the nerve (only tight enough to elicit a twitch). All sutures were within a ½ cm range of each other. The incision was then closed in layers, using 4.0 silk sutures, and the skin closed using sterile autoclips, Topical antibiotic ointment was applied to the sutured incision. All subjects received an analgesic (buprenorphine, 0.05 mg/kg, s.c.) immediately before and 6 hours after surgery. Each rat was monitored until awake and moving freely around the recovery chamber. Animals were then single-housed for the duration of the study. Rats began their chronic-treatment regimen (all test groups except gabapentin) 1 hour post-op on surgery day.

### 1.3.2 Von Frey Test

Withdrawal from a mechanical stimulus was measured by applying von Frey (VF) filaments of ascending bending force to the plantar surface of the hind paws (ipsilateral and contralateral). A positive response was defined as withdrawal from the von Frey filament. Confirmation of the paw withdrawal threshold (PWT) was tested by assessing the response to the filament above and below the withdrawal response.

Rats were brought to the experimental room and allowed to habituate in the room for one hour prior to testing, and acclimated to the observation chambers for 15 minutes prior to taking PWT measurements.

Pre-operative baseline testing: Prior to surgery, all rats were tested using the VF test. Rats that had an ipsilateral PWT of less than 12 g were excluded from the study. Rats were subsequently balanced and assigned to treatment groups (n=10-11 per group) based on their post-op PWT values.

Post-op testing: On Day 12 post-surgery, rats were injected with vehicle, gabapentin, or test compound and tested 1 hour following administration.

### 1.3.4 Statistical Analysis

Data were analyzed by one-way analysis of variance (ANOVA) followed by Fisher PLSD post-hoc comparisons. An effect was considered significant if p<0.05. Data are presented as the mean±standard error of the mean (S.E.M.).

### 1.4. Results.

### 1.4.1 Baseline Responses

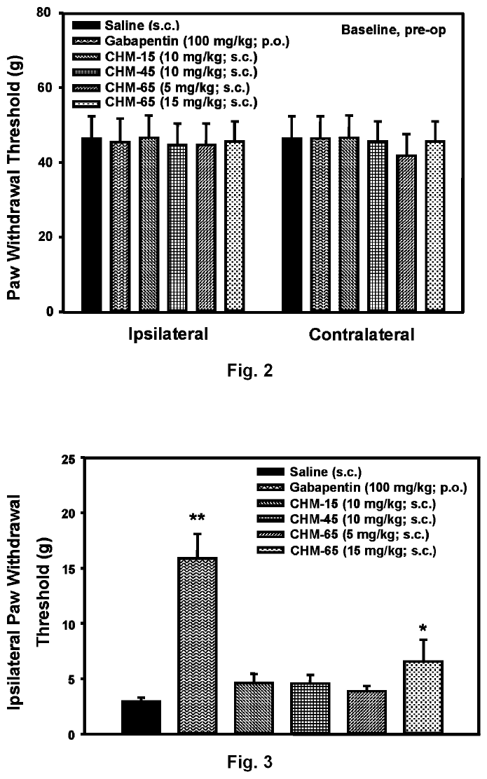

Prior to surgery, all rats were tested using the VF test. Rats that had an ipsilateral PWT of less than 12 g were excluded from the study. Rats were subsequently balanced and assigned to treatment groups (n=11-12 per group) based on their pre-surgery PWT values. One-way ANOVA found no differences between all the different groups assigned to the various treatments (FIG. 2).

### 1.4.2 Effects of Test Compounds on Ipsilateral Paw Withdrawal Threshold

The effects of gabapentin (acute) and Chemedest test compounds (subchronic) on ipsilateral paw withdrawal threshold in sciatic-ligated rats are shown in FIG. 3, Signs of sedation or toxicity were not observed during pretreatment or testing. One-way ANOVA showed a significant main effect of treatment. Post hoc analysis indicated that compared to vehicle, gabapentin and CHM-65 (15 mg/kg) significantly increased ipsilateral paw withdrawal threshold.

### Example 2. Evaluation of the Analgesic Properties of Compounds in Rat Models of Neuropathic Pain (Chung Model)

The study was designed to evaluate the analgesic efficacy of test compounds in the rat Chung model of neuropathic pain.

### 2.1 Animals

Male Sprague Dawley rats (100-125 g) from Harlan (Indianapolis, Ind.) were used in the study. Upon receipt, rats were assigned unique identification numbers and were group housed with 3 rats per cage in polycarbonate cages with micro-isolator filter tops. All rats were examined, handled, and weighed prior to initiation of the study to assure adequate health and suitability. During the course of the study, 12/12 light/dark cycles were maintained, with lights on at 7:00 am EST. The room temperature was maintained between 20 and 23° C. with a relative humidity maintained around 50%. Chow and water were provided ad libitum for the duration of the study.

### 2.2 Test Compounds

The following compounds were used for this study. The vehicles were administered at a dose volume equivalent to the test compound administered.

Reference compound: Gabapentin (100 mg/kg; TRC, Lot No. 1-SWM-154-1) was dissolved in saline and administered acutely on test day (day 8 or 12) 1 hour prior to testing, at a dose volume of 1 ml/kg, p.o.

Test compounds: CHM-65 (5, 15 and 25 mg/kg, Q.D., Lot n/a) was dissolved in sterile injectable saline and administered subcutaneously on days 1, 3, 5 and 8 with the first administration (day 1) occurring 1 hour post-op. On day 8, compound was administered 1 hour prior to testing. The dose volume was 3 ml/kg.

CHM-36 (20 mg/kg, Q.D., Lot n/a) was dissolved in sterile injectable saline and administered subcutaneously on days 1, 3, 5 and 8 with the first administration (day 1) occurring 1 hour post-op. On day 8, compound was administered 1 hour prior to testing. The dose volume was 5 ml/kg.

Artemin (CHMB-1) (0.5 mg/kg, Q.D., Lot n/a) was administered subcutaneously on days 1, 3, 5, 8, 10 and 12 with the first administration (day 1) occurring 1 hour post-op. On day 12, compound was administered 1 hour prior to testing. The dose volume was 0.5 ml/kg,

### 2.3 Methods

### 2.3.1 Spinal Nerve Ligation—Chung

Under general anesthesia with continuous inhalation of isoflurane, surgery was performed with aseptic procedures in surgery unit. Sterile ophthalmic ointment was used to lubricate the eyes. Animals were observed continuously for the level of anesthesia, testing for the animal's reflex response to tail or paw pinch and closely monitoring the animal's breathing. A heating pad was used to maintain body temperature at 37° C. while the animals recovered from anesthesia. The skin at the area of the lower lumber and sacral level of the rat was shaved and disinfected with betadine and alcohol. A left longitudinal incision at the level next to the vertebral column was made and the left paraspinal muscles were separated. The transverse process of L6 was removed and nearby connective tissue cleaned to expose L5 and L6 spinal nerves. After the nerves were isolated and clearly visualized, 4-0 silk threads were used to ligate the left L5. The muscles were sutured with 4-0 silk threads and the wound closed by staples. All rats received an analgesic (buprenorphine, 0.05 mg/kg, s.c.) immediately before and 6 hours after surgery. Each rat was monitored until awake and moving freely around the recovery chamber. Animals were then single-housed for the duration of the study. Rats began their chronic-treatment regimen (all test groups except gabapentin) 1 hour post-op on surgery day.

### 2.3.2 Von Frey Test

Withdrawal from a mechanical stimulus was measured by applying von Frey (VF) filaments of ascending bending force to the plantar surface of the hind paws (ipsilateral and contralateral). A positive response was defined as withdrawal from the von Frey filament. Confirmation of the paw withdrawal threshold (PWT) was tested by assessing the response to the filament above and below the withdrawal response.

Rats were brought to the experimental room and allowed to habituate in the room for one hour prior to testing, and acclimated to the observation chambers for 15 minutes prior to taking PWT measurements.

Pre-operative baseline testing: Prior to surgery, all rats were tested using the VF test. Rats that had an ipsilateral PWT of less than 12 g were excluded from the study. Rats were subsequently balanced and assigned to treatment groups (n=10-12 per group) based on their pre-op PWT values.

Post-op testing: On Day 8 or 12 post-surgery, rats were injected with vehicle, gabapentin, or test compound and tested 1 hour following administration.

### 2.3.3 Statistical Analysis

Data were analyzed by one-way analysis of variance (ANOVA) followed by Fisher PLS D post-hoc comparisons. An effect was considered significant if p<0.05. Data are presented as the mean±standard error of the mean (S.E.M.).

### 2.4, Results (CHM-36)

### 2.4.1 Baseline Responses

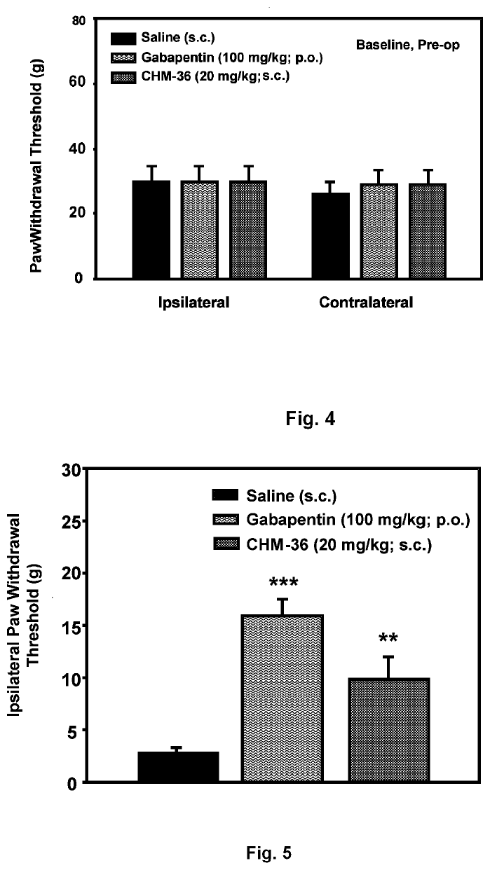

Prior to surgery, all rats were tested using the VF test. Rats that had an ipsilateral PWT of less than 12 g were excluded from the study. Rats were subsequently balanced and assigned to treatment groups (n=12 per group) based on their pre-surgery PWT values. One-way ANOVA found no differences between all the different groups assigned to the various treatments (FIG. 4).

### 2.4.2 Effects of Test Compound on Ipsilateral Paw Withdrawal Threshold

The effects of gabapentin (acute) and Chemedest test compound (subchronic) on ipsilateral paw withdrawal threshold in spinal nerve-ligated rats are shown in FIG. 5. Signs of sedation or toxicity were not observed during pretreatment or testing. One-way ANOVA showed a significant main effect of treatment. Post hoc analysis indicated that compared to vehicle, gabapentin and CHM-36 (20 mg/kg) significantly increased ipsilateral paw withdrawal threshold.

### 2.5, Results (Artemin, CHMB-1)

### 2.5.1 Baseline Responses

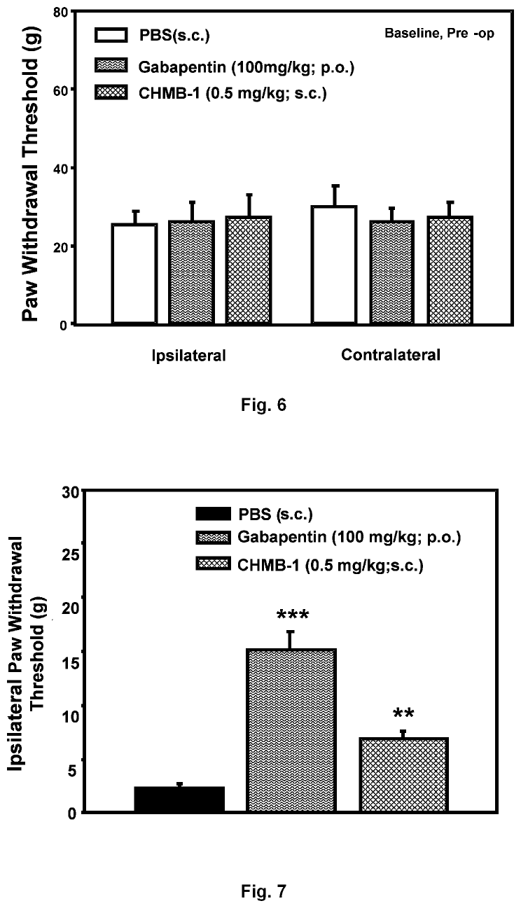

Prior to surgery, all rats were tested using the VF test. Rats that had an ipsilateral PWT of less than 12 g were excluded from the study. Rats were subsequently balanced and assigned to treatment groups (n=10-12 per group) based on their pre-surgery PWT values. One-way ANOVA found no differences between all the different groups assigned to the various treatments (FIG. 6).

### 2.5.2 Effects of Test Compound on Ipsilateral Paw Withdrawal Threshold

The effects of gabapentin (acute) and artemin (CHMB-1) on ipsilateral paw withdrawal threshold in spinal nerve-ligated rats are shown in FIG. 7. Signs of sedation or toxicity were not observed during pretreatment or testing. One-way ANOVA showed a significant main effect of treatment. Post hoc analysis indicated that compared to vehicle, gabapentin and CHMB-1 (0.5 mg/kg) significantly increased ipsilateral paw withdrawal threshold.

### REFERENCES

Airaksinen et al, 2002 Airaksinen, and Saarma. (2002) The GDNF family: signalling, biological functions and therapeutic value, Nat Rev Neurosci. 3:383-94.
Baloh et al, 1998 Baloh, R. H., Tansey, M. G., Lampe, P. A., Fahrner, T. J., Enomoto, H., Simburger, K. S., Leitner, M. L., Araki, T., Johnson, E. M., Jr., and Milbrandt, J. (1998). Artemin, a novel member of the GDNF ligand family, supports peripheral and central neurons and signals through the GFRalpha3-RET receptor complex. Neuron 21: 1291-1302,
Gardell et al, 2003 Gardell L. R., Wang R., Ehrenfels C., et al. (2003) Multiple actions of systemic artemin in experimental neuropathy. Nat Med. 2003; 9: 1383-1389,
Ossipov, 2011 Ossipov, M. H. (2011) Growth Factors and Neuropathic Pain, Pain and Headache Reports, 15: 185-192,
Wang, et al 2003 Wang, R., King, T., Ossipov, M. H., et al, Persistent restoration of sensory function by immediate or delayed systemic artemin after dorsal root injury, Nat. Neusrosci., 11: 488-496.

1. A substance or a pharmaceutically acceptable salt thereof for use in the method of treating or preventing peripheral neuropathy in a subject determined to be in need thereof comprising: topically administering to the subject an anti-peripheral neuropathic compound acting as GFR α 3 type receptor agonist and having the following compound structure: 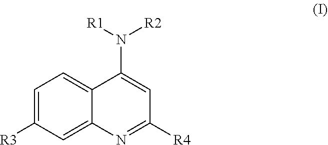
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3 is independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl and R4 is selected from the group consisting of H, alkyl, aryl, aralkyl, hydroxyl; or a pharmaceutically acceptable salt thereof.

2. A substance or a pharmaceutically acceptable salt thereof for use according to claim 1 wherein compound having one of the following compound structures 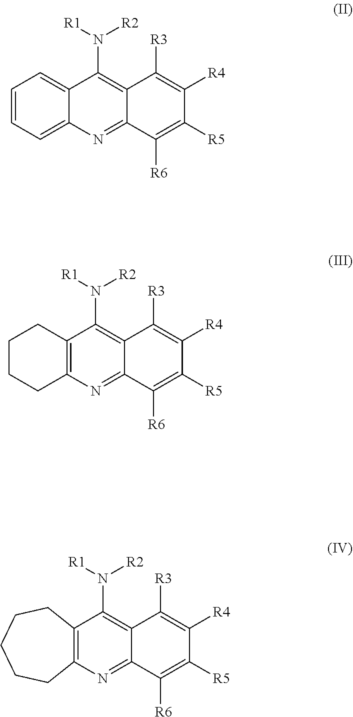
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, R4, R5, and R6 are independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl; or a pharmaceutically acceptable salt thereof.

3. A substance or a pharmaceutically acceptable salt thereof for use according to claim 1 wherein compound having following compound of a structure 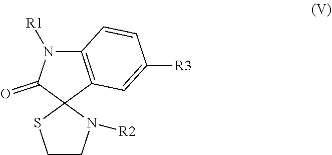
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, is independently selected from H, fluorine, chlorine, bromine, iodide, alkyl, aryl, aralkyl, acyl, alkoxy, alkoxycarbonyl, aryloxycarbonyl, aralkoxycarbonyl, carbamoyl, alkylcarbamoyl, and dialkylcarbamoyl; or a pharmaceutically acceptable salt thereof.

4. A substance or a pharmaceutically acceptable salt thereof for use according to claim 1 wherein comprising the anti-peripheral neuropathic compound acting as GFR α 3 type receptor agonist having one of the following compound structures:

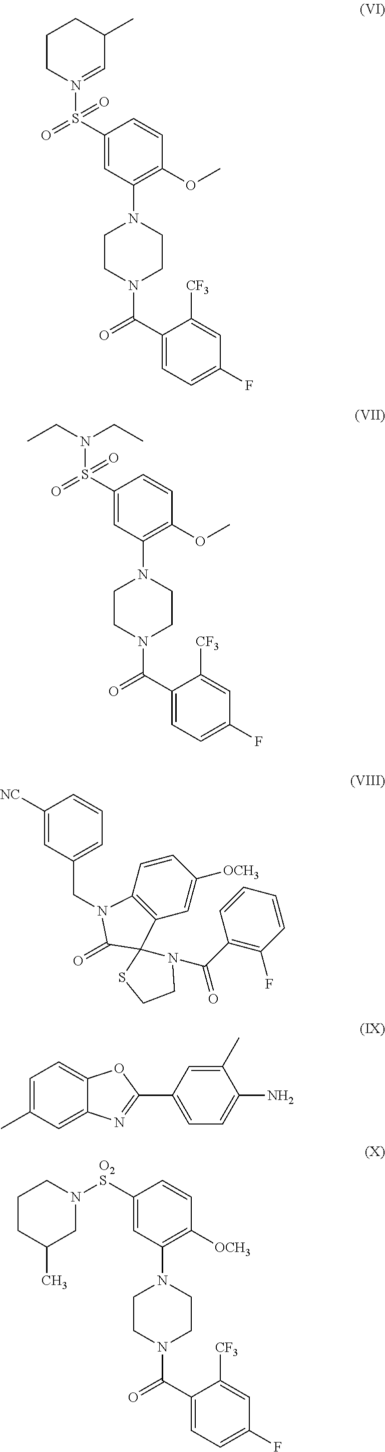

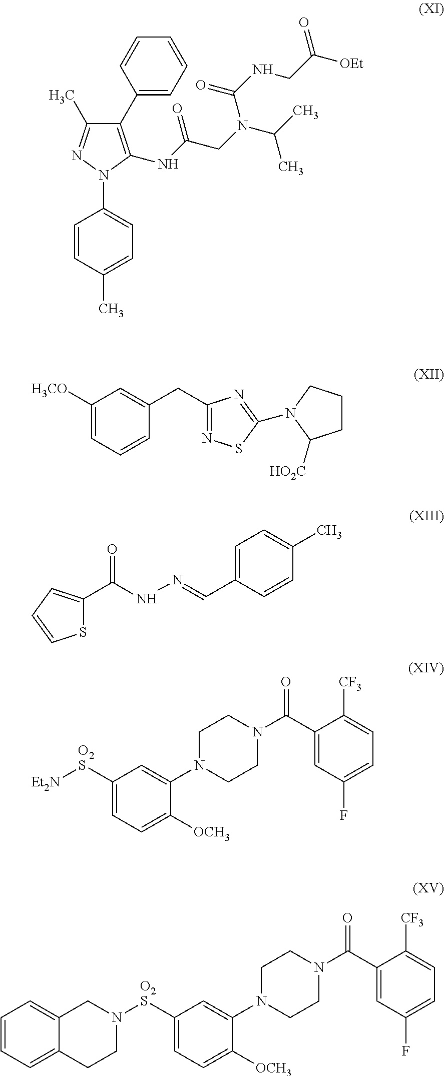

5. A method to treat or prevent peripheral neuropathy in a subject determined to be in need thereof said method comprising a step of topically administering to the subject an anti-peripheral neuropathic compound acting as GFR α 3 type receptor agonist and having the following compound structure: 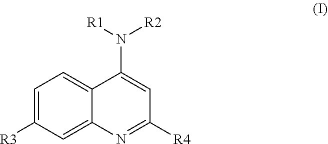
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3 is independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl and R4 is selected from the group consisting of H, alkyl, aryl, aralkyl, hydroxyl; or a pharmaceutically acceptable salt thereof.

6. The method according to claim 5 wherein the compound has one of the following compound structures 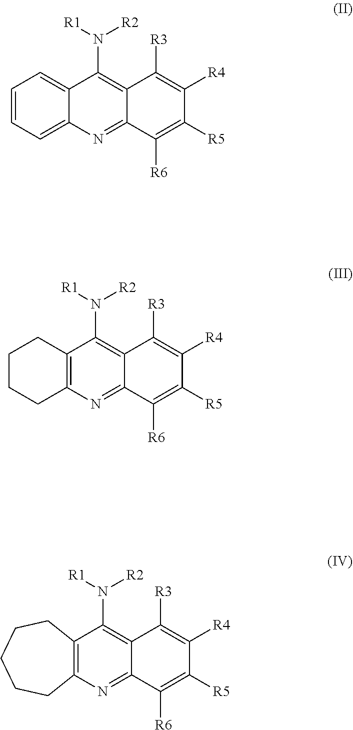
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, R4, R5, and R6 are independently selected from H, fluorine, chlorine, bromine, iodide, alkyl, aryl, aralkyl, acyl, alkoxycarbonyl, aryloxycarbonyl, aralkoxycarbonyl, carbamoyl, alkylcarbamoyl, and dialkylcarbamoyl; or a pharmaceutically acceptable salt thereof.

7. The method of claim 5 wherein the compound has the following compound structure 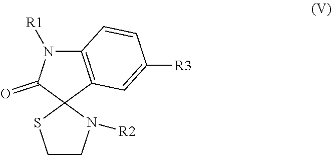
wherein: R1 and R2 are independently selected from the group consisting of H, alkyl, aryl, aralkyl, acyl, alkoxycarbonyl, aryloxycarbonyl, aralkoxycarbonyl, carbamoyl, alkylcarbamoyl, and dialkylcarbamoyl, aminoalkyl, aminoalaryl; R3, is independently selected from H, fluorine, chlorine, bromine, iodide, alkyl, aryl, aralkyl, acyl, alkoxy, alkoxycarbonyl, aryloxycarbonyl, aralkoxycarbonyl, carbamoyl, alkylcarbamoyl, and dialkylcarbamoyl; or a pharmaceutically acceptable salt thereof.

8. The method according to claim 5 wherein the anti-peripheral neuropathic compound acting as GFR α 3 type receptor agonist has one of the following compound structures: 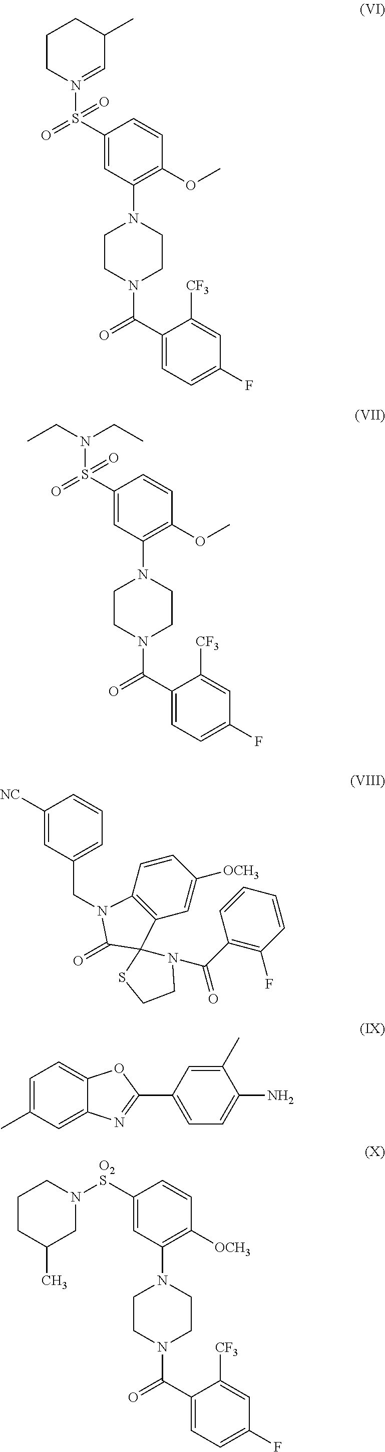 [chemical structure]
